(19) United States
(12) Patent Application Publication
(10) Pub. No.: US 2026/0053844 A1
(43) Pub. Date: Feb. 26, 2026

(54) COMPOSITIONS AND METHODS FOR WEIGHT LOSS AND SATIETY IMPROVEMENT

(71) Applicant: Shaklee Corporation, Miami, FL (US)

(72) Inventors: Jennifer Anderson, Springville, UT (US); Grace Ashton, Springville, UT (US); Jason Pickering, Springville, UT (US); Sarah Eliaso, Springville, UT (US)

(21) Appl. No.: 19/306,667

(22) Filed: Aug. 21, 2025

Related U.S. Application Data
(60) Provisional application No. 63/685,596, filed on Aug. 21, 2024.
(60) Provisional application No. 63/700,136, filed on Sep. 27, 2024.

(51) Int. Cl. A61K 31/736; A23L 2/56; A23L 2/60; A61K 9/00; A61K 33/00; A61K 36/752; A61K 36/81; A61K 36/888; A61K 47/10; A61P 3/04

(52) U.S. Cl. CPC A61K 31/736; A23L 2/56; A23L 2/60; A61K 9/0095; A61K 33/00; A61K 36/752; A61K 36/81; A61K 36/888; A61K 47/10; A61P 3/04

(57) ABSTRACT

Provided herein are compositions, formulations, and methods of treatment for weight loss and satiety improvement for a subject in need thereof, including a human subject in need thereof. The compositions may be used to improve satiety, insulin sensitivity and the microbiome in a subject in need thereof. The composition may further used for weight management and/or in mammals, including humans.

### CROSS REFERENCE TO RELATED APPLICATIONS

This application claims the benefit of U.S. Provisional Application No. 63/700,136, filed on Sep. 27, 2024, and U.S. Provisional Application No. 63/685,596, filed on Aug. 21, 2024. The entirety of each of the foregoing applications is hereby incorporated herein by reference.

### BACKGROUND

Individual weight management is one of the greatest problems of modern society. The health consequences of increasing obesity range from increased risk of premature death to serious chronic conditions that reduce the overall quality of life. To mitigate this problem, the use of behavior and lifestyle modification in weight management is commonly used along with surgical intervention. More recently, behavior and lifestyle modification have been used in combination with low-calorie diets, medical nutrition therapy, nutrition education, exercise programs, monitoring, pharmacological agents, and social support to promote weight loss, and as a component of weight maintenance programs.

Provided herein are compositions that may be used for weight management. It is a further object of the present invention to provide compositions and methods that have a synergistic effect on humans to reduce or modulate weight gain, improve satiety, regulate insulin sensitivity, promote GLP1 production, and/or improve the microbiome to a subject in need thereof.

### SUMMARY

Provided herein, inter alia, are compositions comprising at least two of the following agents including a bulking agent; a satiety modifying agent; a prebiotic agent; and wherein the composition is formulated in a powder.

Provided herein, inter alia, is also a method of inducing satiety in a mammal in need thereof, comprising administering a composition comprising at least two of the following agents including a bulking agent comprising glucomannan optionally derived from konjac root; a satiety modifying agent optionally comprising lemon bioflavonoids; a prebiotic agent comprising potato starch; wherein the individual agents are present in an amount from about 0.01 g to about 3.0 g, from about 0.01 g to about 4.5 g, from about 0.01 g to about 6.0 g, from about 0.01 g to about 7.5 g, from about 0.01 g to about 8.0 g, from about 0.01 g to about 9.0 g, or from about 0.01 g to about 25.0 g; and wherein the individual agents are formulated in a powder and mixed with water to form a solution or a suspension prior to administration.

In one embodiment is a composition comprising a bulking agent; a satiety modifying agent; and a prebiotic agent; wherein the composition is formulated in a powder.

In another embodiment, a composition suitable for oral administration to a human subject in need thereof is described herein. The composition comprising potato starch, glucomannan optionally derived from konjac root, chromium, green olive leaf, grape see extract, green tea extract, grapefruit extract, bilberry extract, white grape extract, red grape extract, and citrus hesperidin. In an embodiment, the composition further includes a polyphenol or polyphenol blend.

In an embodiment, the composition promotes GLP1 production in vivo in a mammal. In another embodiment, the composition promotes collagen integrity in the mammal.

In an embodiment, the method is used to improve satiety, insulin sensitivity and microbiome. In another embodiment, the method is used to suppress appetite in the mammal. In another embodiment, the method is used to curb cravings. In another embodiment, the method is further used for weight management and to induce weight loss in mammals in need thereof.

### DETAILED DESCRIPTION

### Definitions

All publications, patents and patent applications cited herein, whether supra or infra, are hereby incorporated by reference in their entirety to the same extent as if each individual publication, patent, or patent application were specifically and individually indicated to be incorporated by reference.

The term “composition” refers to a mixture of materials which comprise the composition, as well as reaction products and decomposition products formed from the materials of the composition.

The terms “comprising,” “including,” “having” and their derivatives, are not intended to exclude the presence of any additional component, step, or procedure, whether or not the same is specifically disclosed. In order to avoid any doubt, all compositions claimed through use of the term “comprising” may include any additional additive, adjuvant, or compound, whether polymeric or otherwise, unless stated to the contrary.

The term “or,” unless stated otherwise, refers to the listed members individually as well as in any combination. Use of the singular includes use of the plural and vice versa.

The terms “a,” “an,” “the” and similar referents used in the context of describing the inventive features (especially in the context of the following claims) are to be construed to cover both the singular and the plural, unless otherwise indicated herein or clearly contradicted by context. Thus, for example, reference to a “starch” may include one, two or more starches.

The term “prebiotic” is intended to mean a selectively fermented ingredient that allows specific changes, both in the composition and/or activity in the gastrointestinal microflora flora that confers benefits upon host wellbeing and health. Prebiotics act in the colon and produce changes in the microbial flora which affect energy metabolism and gut peptides involved in satiation (GLP1, GLP2). These have a long-acting affect.

The term “dietary fiber” is intended to mean indigestible portions of food derived from plants which comprise soluble and insoluble fibers.

Unless otherwise stated, all percentages, ratios, parts, and amounts used and described herein are by weight.

Numbers, percentages, ratios, or other values stated herein may include that value, and also other values that are about or approximately the stated value, as would be appreciated by one of ordinary skill in the art. A stated value should therefore be interpreted broadly enough to encompass values that are at least close enough to the stated value to perform a desired function or achieve a desired result, and/or values that round to the stated value. The stated values include at least the variation to be expected in a typical manufacturing process, and may include values that are within 25%, 15%, 10%, within 5%, within 1%, etc. of a stated value.

### Compositions

Provided herein are compositions used to improve satiety, insulin sensitivity, microbiome and/or gut health. The composition is further used for weight management in mammals.

In an embodiment, composition includes any two of the agents including a bulking agent, a satiety modifying agent, or a prebiotic agent. In an embodiment, the composition is formulated in a powder. In an embodiment, the powder is further formulated with water for human consumption.

In an embodiment, the bulking agent comprises glucomannan, cellulose, modified cellulose, hydrogel, psyllium, flax seed, pectin, soluble fiber. In another embodiment, the soluble fiber is tapioca.

In an embodiment, the satiety modifying agent comprises lemon bioflavonoid, potato protein, mulberry extract, flavanones, chromium, yeast hydrolysate, lemon verbena, hibiscus, or a combination thereof.

In an embodiment, the prebiotic agent comprises resistant potato starch, Akkermansia Muciniphila probiotic, pomegranate extract, or a combination thereof.

In an embodiment, the composition preferably acts mechanistically to suppress appetite.

In an embodiment, the bulking agent comprises glucomannan, cellulose, modified cellulose, hydrogel, psyllium, flax seed, pectin, soluble fiber, or a combination thereof.

In an embodiment, the bulking agent comprises glucomannan, optionally obtained from konjac root. Glucomannan acts primarily in the stomach by suppressing appetite by gel formation in the stomach, causing distension and activating mechanoreceptors that signal increased satiety and fullness.

In an embodiment, the satiety modifying agent promotes satiety factors including but not limited to an increase in ghrelin, leptin, GLP-1, CCK or PYY; or an ingredient which promotes satiety via increasing insulin sensitivity or glucose control.

In an embodiment, the satiety modifying agent increases ghrelin, leptin, GLP-1, CCK, PYY or a combination thereof in vivo in the human subject following administration to a human subject.

In an embodiment, the satiety modifying agent preferably acts mechanistically to suppress appetite. In another embodiment, the satiety modifying agent includes dietary fiber.

In an embodiment, the prebiotic agent is used to modify the microbiome. In another embodiment, the prebiotic agent functions, like a prebiotic fiber, resistant insoluble starch or as an Akkermansia probiotic, which propagates the growth of Akkermansia muciniphila in vivo.

In an embodiment, the prebiotic is resistant potato starch, Akkermansia Muciniphila probiotic, or pomegranate extract. In another embodiment, the prebiotic promotes the growth of Akkermansia muciniphila in vivo in the human subject following administration to the human subject. In another embodiment, the prebiotic promotes the growth of Bifidobacterium in vivo in the human subject following administration to the human subject. In another embodiment, the Bifidobacterium comprises Bifidobacterium bifidum, Bifidobacterium infantis, Bifidobacterium longum, or a combination thereof.

In an embodiment, the prebiotic is producible by the probiotic bacterial strain by reverse enzyme reaction. In an embodiment, the prebiotic agent comprises one or more additional microbial strains. In another embodiment, the prebiotic agent is selected from inulin, fructooligosaccharides (FOS), galactooligosaccharides (GOS), α-gluco-oligosaccharides, a low gas producing prebiotic, or a combination thereof.

In an embodiment, the composition further comprises one or more optional agents comprising polyphenol blend, amino acids, amino acid metabolites, co-enzyme complex, elemental complex, chelating agents or flavoring agents. In another embodiment, the coenzyme complex comprises one or more cofactor selected from coenzyme Q10, NADH, NADPH, coenzyme R or B-complex vitamins.

In an embodiment, the composition comprises a peach flavoring, mango flavoring, or a combination thereof.

In an embodiment, the flavoring agent is present in an amount of about 1 to 10 wt. %. In another embodiment, the flavoring agent is present in an amount of about 1 to 8 wt. %. In another embodiment, the flavoring agent is present in an amount of about 2 to 8 wt. %. In another embodiment, the flavoring agent is present in an amount of about 3 to 8 wt. %.

In an embodiment, the flavoring agent is present in an amount of about 3 to 9 wt. %. In another embodiment, the flavoring agent is present in an amount of about 4 to 8 wt. %. In another embodiment, the flavoring agent is present in an amount of about 4 to 6 wt. %. In another embodiment, the flavoring agent is present in an amount of about 4.5 to 6.5 wt. %.

In an embodiment, the flavoring agent is present in an amount of about 4.5 to 7.5 wt. %. In another embodiment, the flavoring agent is present in an amount of about 4.5 to 7 wt. %. In another embodiment, the flavoring agent is present in an amount of about 5 to 6.5 wt. %. In another embodiment, the flavoring agent is present in an amount of about 5 to 5.5 wt. %.

In an embodiment, the flavoring agent is present in an amount of about 4.5 to 5.5 wt. %. In another embodiment, the flavoring agent is present in an amount of about 4.8 to 5.3 wt. %. In another embodiment, the flavoring agent is present in an amount of about 5 to 5.3 wt. %. In another embodiment, the flavoring agent is present in an amount of about 5.1 to 5.3 wt. %.

In an embodiment, the mango flavoring agent is present in an amount of about 4.5 to 5.5 wt. %. In another embodiment, the mango flavoring agent is present in an amount of about 5 to 5.3 wt. %. In another embodiment, the mango flavoring agent is present in an amount of about 5 to 5.2 wt. %. In another embodiment, the mango flavoring agent is present in an amount of about 5.1 to 5.2 wt. %. In another embodiment, the mango flavoring agent is present in an amount of about 5.2 wt. %.

In an embodiment, the peach flavoring agent is present in an amount of about 4.5 to 5.5 wt. %. In another embodiment, the peach flavoring agent is present in an amount of about 5 to 5.3 wt. %. In another embodiment, the peach flavoring agent is present in an amount of about 5 to 5.2 wt. %. In another embodiment, the peach flavoring agent is present in an amount of about 5.1 to 5.2 wt. %. In another embodiment, the peach flavoring agent is present in an amount of about 5.2 wt. %.

In an embodiment, the composition further comprises minerals including, but not limited to, sodium, magnesium, chromium, iodine, iron, manganese, calcium, copper, fluoride, potassium, phosphorous, molybdenum, selenium, zinc, and combinations thereof.

In an embodiment, the mineral is present in an amount of about 0.001 to 0.005 wt. %. In another embodiment, the mineral is present in an amount of about 0.0015 to 0.004 wt. %. In another embodiment, the mineral is present in an amount of about 0.002 to 0.004 wt. %. In another embodiment, the mineral is present in an amount of about 0.0025 to 0.0035 wt. %.

In an embodiment, the mineral is present in an amount of about 0.0028 to 0.004 wt. %. In another embodiment, the mineral is present in an amount of about 0.003 to 0.0035 wt. %.

In an embodiment, the mineral is chromium present in an amount of about 0.0031 to 0.0035 wt. %. In another embodiment, the chromium is present in an amount of about 0.0032 wt. %.

In an embodiment, the composition further comprises antioxidants including but not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenol, a blend of polyphenol, flavonoids, and combinations thereof.

In an embodiment, the citric acid is present in an amount of about 3 to 9 wt. %. In another embodiment, the citric acid is present in an amount of about 4 to 8 wt. %. In another embodiment, the citric acid is present in an amount of about 4 to 6 wt. %. In another embodiment, the citric acid is present in an amount of about 4.5 to 6.5 wt. %.

In an embodiment, the citric acid is present in an amount of about 4.5 to 7.5 wt. %. In another embodiment, the citric acid is present in an amount of about 4.5 to 7 wt. %. In another embodiment, the citric acid is present in an amount of about 5 to 6.5 wt. %.

In an embodiment, the citric acid is present in an amount of about 5.5 to 6.0 wt. %. In another embodiment, the citric acid is present in an amount of about 5.6 to 6.0 wt. %. In another embodiment, the citric acid is present in an amount of about 5.7 to 6.0 wt. %. In another embodiment, the citric acid is present in an amount of about 5.8 to 6.0 wt. %. In another embodiment, the citric acid is present in an amount of about 5.9 wt. %. In another embodiment, the citric acid is present in an amount of about 6.0 wt. %.

In an embodiment, the composition comprises a polyphenol blend. In an embodiment, polyphenol blend is present in an amount of about 0.2 to 2 wt. %. In another embodiment, the citric acid is present in an amount of about 0.5 to 1.8 wt. %. In another embodiment, the citric acid is present in an amount of about 0.7 to 1.8 wt. %.

In an embodiment, polyphenol blend is present in an amount of about 0.8 to 1.8 wt. %. In another embodiment, the polyphenol blend is present in an amount of about 0.9 to 1.8 wt. %. In another embodiment, the polyphenol blend is present in an amount of about 1.0 to 1.8 wt. %. In another embodiment, the polyphenol blend is present in an amount of about 1.2 to 1.8 wt. %. In another embodiment, the polyphenol blend is present in an amount of about 1.2 to 1.7 wt. %. In another embodiment, the polyphenol blend is present in an amount of about 1.3 to 1.7 wt. %. In another embodiment, the polyphenol blend is present in an amount of about 1.4 to 1.5 wt. %.

In an embodiment, polyphenol blend is present in an amount of about less than 3 wt. %. In another embodiment, the polyphenol blend is present in an amount of about 2.5 wt. %. In another embodiment, the polyphenol blend is present in an amount of about 2.0 wt. %.

In an embodiment, the composition further comprises phytochemicals including but not limited to cartotenoids, chlorophyll, chlorophyllin, fiber, flavanoids, anthocyamns, cyaniding, delphinidin, malvidin, pelargonidin, peonidin, petunidin, flavanols, catechin, epicatechin, epigallocatechin, epigailocatechingallate, theaflavins, thearubigins, proanthocyanins, flavonols, quercetin, kaempferol, myricetin, isorhamnetin, flavononeshesperetin, naringenin, criodictyol, tangeretin, flavones, apigenin, luteolin, lignans, phytoestrogens, resveratrol, isoflavones, daidzein, genistein, glycitein, soy isoflavones, and combinations thereof.

In an embodiment, the composition further comprises coloring agents. In an embodiment, the coloring agent is present in an amount of about 0.1 to 5 wt. %. In another embodiment, the coloring agent is present in an amount of about 0.1 to 2 wt. %. In another embodiment, the coloring agent is present in an amount of about 0.1 to 1.5 wt. %. In another embodiment, the coloring agent is present in an amount of about 0.1 to 1 wt. %.

In an embodiment, the coloring agent is present in an amount of about 0.1 to 1.1 wt. %. In another embodiment, the coloring agent is present in an amount of about 0.2 to 1.0 wt. %. In another embodiment, the coloring agent is present in an amount of about 0.2 to 0.9 wt. %. In another embodiment, the coloring agent is present in an amount of about 0.3 to 0.9 wt. %. In another embodiment, the coloring agent is present in an amount of about 0.5 to 0.8 wt. %. In another embodiment, the coloring agent is present in an amount of about 0.6 to 0.8 wt. %. In another embodiment, the coloring agent is present in an amount of about 0.7 to 0.8 wt. %. In an embodiment, the coloring agent is present in an amount of about 0.79 wt. %.

In an embodiment, the flavonoid is present in an amount of about 0.5 to 10 wt. %. In another embodiment, the flavonoid is present in an amount of about 1 to 10 wt. %. In another embodiment, the flavonoid is present in an amount of about 2 to 10 wt. %. In another embodiment, the flavonoid is present in an amount of about 2 to 8 wt. %.

In an embodiment, the flavonoid is present in an amount of about 2 to 7 wt. %. In another embodiment, the flavonoid is present in an amount of about 2.5 to 6 wt. %. In another embodiment, the flavonoid is present in an amount of about 3 to 6 wt. %. In another embodiment, the flavonoid is present in an amount of about 3 to 5 wt. %. In an embodiment, the flavonoid is a lemon flavonoid present in an amount of about 3 to 4 wt. %.

In an embodiment, the composition further comprises additional components comprising silicon dioxide, additives, xanthan gum, debittering agent, maltodextrin, gum acacia, natural flavors, modified food starch, green olive leaf extract, grape seed extract, pomegranate fruit extract, green tea leaf extract, grapefruit fruit extract, bilberry fruit extract, white grapefruit fruit extract, red grapefruit fruit extract, or hesperidin. In an embodiment, the composition further comprises sweeteners comprising artificial or natural sweeteners.

In an embodiment, the sweeteners are stevia, sucralose, sorbitol, xylitol, luo han guo, purified stevia leaf extract, serythritol, advantame, steviol glycosides, acesulfame potassium, saccharin, neotame or aspartame.

In an embodiment, the silicon dioxide is present in an amount of about 1 to 10 wt. %. In another embodiment, the silicon dioxide is present in an amount of about 1 to 8 wt. %. In another embodiment, the silicon dioxide is present in an amount of about 2 to 8 wt. %. In another embodiment, the silicon dioxide is present in an amount of about 2 to 7 wt. %.

In an embodiment, the silicon dioxide is present in an amount of about 1.5 to 5 wt. %. In another embodiment, the silicon dioxide is present in an amount of about 1.5 to 3.5 wt. %. In another embodiment, the silicon dioxide is present in an amount of about 1.5 to 2.5 wt. %. In another embodiment, the silicon dioxide is present in an amount of about 2.0 wt. %.

In an embodiment, the xanthan gum is present in an amount of about 0.5 to 5 wt. %. In another embodiment, the xanthan gum is present in an amount of about 0.5 to 2.5 wt. %. In another embodiment, the xanthan gum is present in an amount of about 0.5 to 2.0 wt. %. In another embodiment, the xanthan gum is present in an amount of about 0.5 to 1.5 wt. %. In another embodiment, the xanthan gum is present in an amount of about 1.5 wt. %.

In an embodiment, the sweetener is present in an amount of about 0.5 to 5 wt. %. In another embodiment, the sweetener is present in an amount of about 0.5 to 2.5 wt. %. In another embodiment, the sweetener is present in an amount of about 0.5 to 2.0 wt. %. In another embodiment, the sweetener is present in an amount of about 0.5 to 1.6 wt. %. In another embodiment, the sweetener is present in an amount of about 0.8 to 1.6 wt. %. In another embodiment, the sweetener is present in an amount of about 1 to 1.5 wt. %. In another embodiment, the sweetener is present in an amount of about 1.3 to 1.5 wt. %. In another embodiment, the sweetener is present in an amount of about 1.4 wt. %.

In an embodiment, the debittering agent is present in an amount of about 0.01 to 0.05 wt. %. In another embodiment, the mineral is present in an amount of about 0.01 to 0.03 wt. %. In another embodiment, the mineral is present in an amount of about 0.01 to 0.02 wt. %. In another embodiment, the mineral is present in an amount of about 0.015 to 0.02 wt. %. In an embodiment, the debittering agent is present in an amount of about 0.015 to 0.016 wt. %. In an embodiment, the debittering agent is present in an amount of about 0.015 wt. %. In an embodiment, the debittering agent is present in an amount of about 0.016 wt. %.

In an embodiment, the composition is gluten free. In another embodiment, the composition is dairy free.

In an embodiment, the composition is sugar free. In another embodiment, the composition contains no added sugars.

In an embodiment, the composition is low fermentable oligosaccharides, disaccharides, monosaccharides and polyols (FODMOP) or suitable for a human subject on a low FODMOP diet.

In an embodiment, the composition is formulated with water to form an aqueous solution or a suspension. In another embodiment, the water is present is an amount of about 1 to about 24 fluid ounces in the aqueous solution or suspension. In another embodiment, the water is present is an amount of about 25 mL to about 250 mL in the aqueous solution or suspension.

In an embodiment, the composition is formulated as a powder, capsule, or drink. In another embodiment, the composition is administered to a human subject as a powder, solution, suspension, capsule, drink mix, or drink.

In an embodiment, the bulking agent is present in an amount of about 0.01 g to about 9 g. In another embodiment, the bulking agent is present in an amount of about 0.05 g to about 9 g. In another embodiment, the bulking agent is present in an amount of about 0.1 g to about 9 g. In another embodiment, the bulking agent is present in an amount of about 0.5 g to about 9 g. In another embodiment, the bulking agent is present in an amount of about 1 g to about 9 g. In another embodiment, the bulking agent is present in an amount of about 5 g to about 9 g.

In yet another embodiment, the bulking agent is present in an amount of about 0.01 g to about 25 g. In another embodiment, the bulking agent is present in an amount of about 0.05 g to about 25 g. In another embodiment, the bulking agent is present in an amount of about 0.1 g to about 25 g. In another embodiment, the bulking agent is present in an amount of about 0.5 g to about 25 g. In another embodiment, the bulking agent is present in an amount of about 1 g to about 25 g. In another embodiment, the bulking agent is present in an amount of about 5 g to about 25 g.

In an embodiment, the bulking agent is present in an amount of about 1 to 30 wt. %. In another embodiment, the bulking agent is present in an amount of about 1 to 25 wt. %. In another embodiment, the bulking agent is present in an amount of about 1 to 20 wt. %. In another embodiment, the bulking agent is present in an amount of about 5 to 20 wt. %.

In an embodiment, the bulking agent is present in an amount of about 5 to 19 wt. %. In another embodiment, the bulking agent is present in an amount of about 6 to 18 wt. %. In another embodiment, the bulking agent is present in an amount of about 6 to 17 wt. %. In another embodiment, the bulking agent is present in an amount of about 6 to 16 wt. %.

In an embodiment, the bulking agent is present in an amount of about 8 to 19 wt. %. In another embodiment, the bulking agent is present in an amount of about 8 to 18 wt. %. In another embodiment, the bulking agent is present in an amount of about 9 to 17 wt. %. In another embodiment, the bulking agent is present in an amount of about 10 to 16 wt. %.

In an embodiment, the bulking agent is present in an amount of about 11 to 18 wt. %. In another embodiment, the bulking agent is present in an amount of about 12 to 18 wt. %. In another embodiment, the bulking agent is present in an amount of about 12 to 17 wt. %. In another embodiment, the bulking agent is present in an amount of about 12 to 16 wt. %.

In an embodiment, the bulking agent is present in an amount of about 13 to 17 wt. %. In another embodiment, the bulking agent is present in an amount of about 14 to 17 wt. %. In another embodiment, the bulking agent is present in an amount of about 15 to 17 wt. %.

In an embodiment, the bulking agent is present in an amount of about 14.5 to 17.5 wt. %. In another embodiment, the bulking agent is present in an amount of about 14.5 to 17 wt. %. In another embodiment, the bulking agent is present in an amount of about 14.5 to 16 wt. %.

In an embodiment, the bulking agent is present in an amount of about 14 to 16 wt. %. In another embodiment, the bulking agent is present in an amount of about 14 to 15.9 wt. %. In another embodiment, the bulking agent is present in an amount of about 14.5 to 15.9 wt. %. In another embodiment, the bulking agent is present in an amount of about 14.8 to 15.9 wt. %. In another embodiment, the bulking agent is present in an amount of about 14.9 to 15.9 wt. %.

In an embodiment, the bulking agent is present in an amount of about 15 to 16 wt. %. In another embodiment, the bulking agent is present in an amount of about 15 to 15.9 wt. %. In another embodiment, the bulking agent is present in an amount of about 15.1 to 15.9 wt. %. In another embodiment, the bulking agent is present in an amount of about 15.2 to 15.9 wt. %. In another embodiment, the bulking agent is present in an amount of about 15.3 to 15.9 wt. %. In another embodiment, the bulking agent is present in an amount of about 15.4 to 15.9 wt. %. In another embodiment, the bulking agent is present in an amount of about 15.5 to 15.9 wt. %. In another embodiment, the bulking agent is present in an amount of about 15.6 to 15.9 wt. %.

In an embodiment, the satiety modifying agent is present in an amount of about 0.01 g to about 9 g. In another embodiment, the satiety modifying agent is present in an amount of about 0.05 g to about 9 g. In another embodiment, the satiety modifying agent is present in an amount of about 0.1 g to about 9 g. In another embodiment, the satiety modifying agent is present in an amount of about 0.5 g to about 9 g. In another embodiment, the satiety modifying agent is present in an amount of about 1 g to about 9 g. In another embodiment, the satiety modifying agent is present in an amount of about 5 g to about 9 g.

In yet another embodiment, the satiety modifying agent is present in an amount of about 0.01 g to about 25 g. In another embodiment, the satiety modifying agent is present in an amount of about 0.05 g to about 25 g. In another embodiment, the satiety modifying agent is present in an amount of about 0.1 g to about 25 g. In another embodiment, the satiety modifying agent is present in an amount of about 0.5 g to about 25 g. In another embodiment, the satiety modifying agent is present in an amount of about 1 g to about 25 g. In another embodiment, the satiety modifying agent is present in an amount of about 5 g to about 25 g.

In an embodiment, the prebiotic agent is present in an amount of about 0.01 g to about 9 g. In another embodiment, the prebiotic agent is present in an amount of about 0.05 g to about 9 g. In another embodiment, the prebiotic agent is present in an amount of about 0.1 g to about 9 g. In another embodiment, the prebiotic agent is present in an amount of about 0.5 g to about 9 g. In another embodiment, the prebiotic agent is present in an amount of about 1 g to about 9 g. In another embodiment, the prebiotic agent is present in an amount of about 5 g to about 9 g.

In yet another embodiment, the prebiotic agent is present in an amount of about 0.01 g to about 25 g. In another embodiment, the prebiotic agent is present in an amount of about 0.05 g to about 25 g. In another embodiment, the prebiotic agent is present in an amount of about 0.1 g to about 25 g. In another embodiment, the prebiotic agent is present in an amount of about 0.5 g to about 25 g. In another embodiment, the prebiotic agent is present in an amount of about 1 g to about 25 g. In another embodiment, the prebiotic agent is present in an amount of about 5 g to about 25 g.

In an embodiment, the prebiotic agent is present in an amount of about 20 to 80 wt. %. In another embodiment, the prebiotic agent is present in an amount of about 25 to 75 wt. %. In another embodiment, the prebiotic agent is present in an amount of about 30 to 70 wt. %. In another embodiment, the prebiotic agent is present in an amount of about 30 to 65 wt. %.

In an embodiment, the prebiotic agent is present in an amount of about 40 to 75 wt. %. In another embodiment, the prebiotic agent is present in an amount of about 45 to 70 wt. %. In another embodiment, the prebiotic agent is present in an amount of about 45 to 65 wt. %. In another embodiment, the prebiotic agent is present in an amount of about 50 to 65 wt. %.

In an embodiment, the prebiotic agent is present in an amount of about 50 to 60 wt. %. In another embodiment, the prebiotic agent is present in an amount of about 55 to 60 wt. %. In another embodiment, the prebiotic agent is present in an amount of about 55 to 58 wt. %. In another embodiment, the prebiotic agent is present in an amount of about 55 to 57 wt. %.

In an embodiment, the prebiotic agent is present in an amount of about 56 to 60 wt. %. In another embodiment, the prebiotic agent is present in an amount of about 56 to 59 wt. %. In another embodiment, the prebiotic agent is present in an amount of about 56 to 58 wt. %. In another embodiment, the prebiotic agent is present in an amount of about 56 to 57 wt. %.

In an embodiment, the prebiotic agent is present in an amount of about at least 50 wt. %. In another embodiment, the prebiotic agent is present in an amount of about 53 wt. %. In another embodiment, the prebiotic agent is present in an amount of about 54 wt. %. In another embodiment, the prebiotic agent is present in an amount of about 55 wt. %. In another embodiment, the prebiotic agent is present in an amount of about 56 wt. %.

In an embodiment, the prebiotic agent is present in an amount of greater than about 53 wt. %. In another embodiment, the prebiotic agent is present in an amount of greater than about 54 wt. %. In another embodiment, the prebiotic agent is present in an amount greater than about 54 wt. %. In another embodiment, the prebiotic agent is present in an amount greater than about 55 wt. %. In another embodiment, the prebiotic agent is present in an amount greater than about 56 wt. %.

In an embodiment, the composition can be formulated as a single use sachet comprising a 0.3 ounce single-use dose. In another embodiment, the composition comprises a 9 g single-use dose. In another embodiment, the single-use dose is packaged in a sachet or stick-pack. In another embodiment, the 30 single-use sachets or stick packs are packaged in a box.

In an embodiment, the composition is formulated for use in a mammal. In an embodiment, the composition has at least two of the following agents including a bulking agent comprising glucomannan derived from konjac root, a satiety modifying agent comprising lemon bioflavonoids, or a prebiotic agent comprising potato starch. In an embodiment, the amount of each agent is about 0.01 g to 25 g.

In an embodiment, the composition is suitable for oral administration to a human subject in need thereof comprising potato starch, glucomannan optionally derived from konjac root, chromium, green olive leaf, grape see extract, green tea extract, grapefruit extract, bilberry extract, white grape extract, red grape extract, and citrus hesperidin.

In an embodiment, the composition has a complete absence of organic acids. In another embodiment, the composition has a complete absence of polypeptides. In another embodiment, the composition has no organic acids. In another embodiment, the composition has no polypeptides.

In an embodiment, the composition is suitable for administration to a human subject in need thereof.

In an embodiment, the composition has solnul, mango flavor, peach flavor, debitter, stevia, citric acid, xanthan gum, silicon dioxide, oxxynea powder (polyphenol blend), eriomin, natural berry color, glucomannan, and chromium picolinate powder.

In an embodiment, the composition has solnul, mango flavor SO44, nat peach ZE39225, nat Debittering HTBA NF01, organic rebaudioside A 98%, citric acid, xanthan gum, syloid 244 silicon dioxide, oxxynea powder, criomin, exberry intense pink, glucomannan konjac and chromium picolinate powder. Suitable amount

In an embodiment, solnul (e.g., digestive resistant potato starch) is present in an amount of about 3000 to 3800 mg per serving, the mango flavor is present in an amount of about 300 to 350 mg per serving, the peach flavor is present in amount of about 300 to 350 mg per serving, debitter is present in an amount of about 0.5 to 1.2 mg per serving, stevia is present in amount of about 85 to 95 mg per serving, citric acid is present in amount of about 350 to 400 mg per serving, xanthan gum is present in amount of about 90 to 100 mg per serving, silicon dioxide is present in amount of about 125 to 135 mg per serving, oxxynea powder (polyphenol blend) is present in amount of about 125 to 135 mg per serving, the criomin is present in amount of about 230 to 260 mg per serving, natural berry color is present in amount of about 40 to 60 mg per serving, glucomannan is present in amount of about 800 to 1200 mg per serving, and the chromium picolinate powder is present in amount of about 0.020 to 0.025 mg per serving.

In an embodiment, solnul is present in an amount of about 3500 mg per serving, the mango flavor is present in an amount of about 325 mg per serving, the peach flavor is present in amount of about 325 mg per serving, debitter is present in an amount of about 1 mg per serving, stevia is present in amount of about 90 mg per serving, citric acid is present in amount of about 375 mg per serving, xanthan gum is present in amount of about 95 mg per serving, silicon dioxide is present in amount of about 129 mg per serving, oxxynea powder (polyphenol blend) is present in amount of about 129 mg per serving, the criomin is present in amount of about 250 mg per serving, natural berry color is present in amount of about 50 mg per serving, glucomannan is present in amount of about 1000 mg per serving, and the chromium picolinate powder is present in amount of about 0.023 mg per serving.

In an embodiment, the sum of all the individual ingredients present in the composition is 100 wt. %.

In an embodiment, solnul is present in an amount of about 50 to 60 wt. %, mango flavor is present in an amount of about 4.5 to 5.5 wt. %, peach flavor is present in amount of about 4.5 to 5.5 wt. %, debittering is present in an amount of about 0.0130 to 0.0160 wt. %, organic sweetener is present in amount of about 1.25 to 1.5 wt. %, citric acid is present in amount of about 4.5 to 6.5 wt. %, xanthan gum is present in amount of about 0.5 to 2 wt. %, silicon dioxide is present in amount of about 1.5 to 2.5 wt %, oxxynea powder is present in amount of about 1.25 to 1.5 wt. %, criomin is present in amount of about 2.5 to 4.5 wt. %, natural berry color is present in amount of about 0.5 to 1.0 wt. %, glucomannan konjac is present in amount of about 12 to 18 wt. % and chromium picolinate powder is present in amount of about 0.0025 to 0.004 wt. %.

In an embodiment, solnul is present in an amount of about 55 to 58 wt. %, mango flavor SO44 is present in an amount of about 4.8 to 5.2 wt. %, nat peach ZE39225 is present in amount of about 4.8 to 5.2 wt. %, nat debittering HTBA NF01 is present in an amount of about 0.0150 to 0.0163 wt. %, organic rebaudioside A 98% is present in amount of about 1.38 to 1.45 wt. %, citric acid is present in amount of about 5.0 to 6.2 wt. %, xanthan gum is present in amount of about 1.0 to 1.8 wt. %, syloid 244 silicon dioxide is present in amount of about 1.8 to 2.2 wt. %, oxxynea powder is present in amount of about 1.39 to 1.45 wt. %, criomin is present in amount of about 3.5 to 4.2 wt. %, exberry intense pink is present in amount of about 0.6 to 0.9 wt. %, glucomannan konjac is present in amount of about 15 to 16 wt. % and chromium picolinate powder is present in amount of about 0.0028 to 0.035 wt. %.

In an embodiment, solnul is present in an amount of about 57 wt %, mango flavor SO44 is present in an amount of about 5.2 wt. %, nat peach ZE39225 is present in amount of about 5.2 wt. %, nat debittering HTBA NF01 is present in an amount of about 0.016 wt. %, organic rebaudioside A 98% is present in amount of about 1.43 wt. %, citric acid is present in amount of about 6 wt. %, xanthan gum is present in amount of about 1.51 wt. %, syloid 244 silicon dioxide is present in amount of about 2 wt. %, oxxynea powder is present in amount of about 1.43 wt. %, criomin is present in amount of about 4.0 wt. %, exberry intense pink is present in amount of about 0.8 wt. %, glucomannan konjac is present in amount of about 15.9 wt. % and chromium picolinate powder is present in amount of about 0.0032 wt. %.

### Methods

Provided herein is a method of inducing satiety in a mammal including but not limited to humans. In an embodiment, the method includes administering a composition comprising at least two of the agents to the mammal including a bulking agent, a satiety modifying agent, or a prebiotic agent. In an embodiment, the individual agents are present in an amount of about 0.01 g to about 25.0 g.

In an embodiment, the method is used to improve satiety, insulin sensitivity and microbiome. In another embodiment, the method is used to suppress appetite in the mammal. In another embodiment, the method is used to curb cravings. In another embodiment, the method is further used for weight management and to induce weight loss in mammals in need thereof.

In an embodiment, the method further promotes GLP1 production in vivo in the mammal. In another embodiment, the method further promotes collagen integrity in the mammal in need thereof.

In an embodiment upon administration of the composition, ≥65% of the time the human subject experiences a decrease in hunger within seven days. In another embodiment upon administration of the composition, ≥55% of the time the human subject experiences a decrease in hunger within seven days. In another embodiment upon administration of the composition, ≥50% of the time the human subject experiences a decrease in hunger within seven days.

In an embodiment upon administration of the composition, ≥50% of the time the human subject experiences a reduction in cravings for sugar within seven days after administration. In another embodiment upon administration of the composition, ≥40% of the time the human subject experiences an increase in bowel regularity within seven days after administration. In another embodiment upon administration of the composition, ≥75% of the time the human subject experiences a decrease in snacking within thirty days after administration. In another embodiment upon administration of the composition, ≥55% of the time the human subject experiences a decrease in hunger within thirty days after administration. In another embodiment upon administration of the composition, ≥50% of the time the human subject experiences a reduction in cravings for sugar within thirty days after administration.

In an embodiment upon administration of the composition, the human subject experiences a weight loss of at least 4.1 pounds, at least 4.3 pounds, 4.5 pounds, or 4.7 pounds within thirty days after administration. In another embodiment upon administration of the composition, the human subject experiences a weight loss of at least 2.1%, at least 2.2%, at least 2.3%, at least 2.4%, or at least 2.5% of the subject's total body weight within thirty days after administration.

In an embodiment, the composition is administered daily for at least 7 days, at least 10 days, or at least 14 days. In another embodiment, the composition is administered twice daily for at least 7 days, at least 10 days, or at least 14 days. In another embodiment, the composition is administered daily for at least 30 days, at least 45 days, at least 60 days, or at least 90 days. In another embodiment, the composition is administered twice daily for at least 30 days, at least 45 days, at least 60 days, or at least 90 days. In another embodiment, the composition is administered three or more times daily for at least 30 days, at least 45 days, at least 60 days, or at least 90 days.

In an embodiment, the composition is administered as a regime until effective weight loss is achieved in the human subject in need thereof. In an embodiment, the composition is administered until no negative side effects are experienced by the human subject.

In the compositions and methods described herein, the bulking agent, satiety modifying agent, and prebiotic agent may be provided in the same or different amounts. In one aspect, the ratio of the bulking agent to satiety modifying agent by weight may be from about 0.01 to about 5,000 or from about 5,000 to 0.01 relative to one another. In another aspect, the ratio of the bulking agent to satiety modifying agent by weight may be from about 0.1:10 to about 50:0.1, about 0.5:10 to about 10:0.5, about 1.0:5 to about 5:0.1, about 1:3 to about 3:1, about 1:2 to about 2:1, or about 1:1, relative to one another. In another aspect, the ratio of the satiety modifying agent to prebiotic agent by weight may be from about 0.01 to about 5,000 or from about 5,000 to 0.01 relative to one another. In another aspect, the ratio of the satiety modifying agent to prebiotic agent by weight may be from about 0.1:10 to about 50:0.1, about 0.1:10 to about 50:0.1, about 0.5:10 to about 10:0.5, about 1.0:5 to about 5:0.1, about 1:3 to about 3:1, about 1:2 to about 2:1, or about 1:1, relative to one another. In yet another aspect, the ratio of the bulking modifying agent to prebiotic agent by weight may be from about 0.01 to about 5,000 or from about 5,000 to 0.01 relative to one another. In yet another aspect, the ratio of the bulking agent to prebiotic agent by weight may be from about 0.1:10 to about 50:0.1, about 0.5:10 to about 10:0.5, about 1.0:5 to about 5:0.1, about 1:3 to about 3:1, about 1:2 to about 2:1, or about 1:1, relative to one another.

In some embodiments, the compositions described herein are suitable for oral administration to a human subject in need thereof, and comprise a satiety modifying agent, prebiotic agent, and bulking agent, wherein the ratio of satiety modifying agent to prebiotic agent is about 1:5 to about 1:30 by weight, and wherein the ratio of satiety modifying agent to bulking agent is about 1:1 to about 1:10 by weight. In some embodiments, the compositions described herein are suitable for oral administration to a human subject in need thereof, and comprise a satiety modifying agent, prebiotic agent, and bulking agent, wherein the ratio of satiety modifying agent to prebiotic agent is about 1:10 to about 1:20 by weight, and wherein the ratio of satiety modifying agent to bulking agent is about 1:2 to about 1:8 by weight. In some embodiments, the compositions described herein are suitable for oral administration to a human subject in need thereof, and comprise a satiety modifying agent, prebiotic agent, and bulking agent, wherein the ratio of satiety modifying agent to prebiotic agent is about 1:13 to about 1:15 by weight, and wherein the ratio of satiety modifying agent to bulking agent is about 1:3 to about 1:6 by weight. In some embodiments, the compositions described herein are suitable for oral administration to a human subject in need thereof, and comprise a satiety modifying agent, prebiotic agent, and bulking agent, wherein the ratio of satiety modifying agent to prebiotic agent is about 1:14 to about 1:15 by weight, and wherein the ratio of satiety modifying agent to bulking agent is about 1:4 to about 1:5 by weight. In some embodiments, the compositions described herein are suitable for oral administration to a human subject in need thereof, and comprise a satiety modifying agent, prebiotic agent, and bulking agent, wherein the ratio of satiety modifying agent to prebiotic agent is about 1:14 by weight, and wherein the ratio of satiety modifying agent to bulking agent is about 1:4 by weight.

In one embodiment, about 500 to 1500 mg/serving of glucomannan (derived from konjac root), about 100 to about 500 g lemon bioflavonoid, about 3000 to 4000 mg/per serving resistant potato starch were combined to form a mixture. Additional ingredients comprising mango flavor, peach flavor, debitter, Stevia, citric acid, xanthan gum, silicon dioxide, Oxxynea (polyphenol blend), Eriomin (lemon bioflavonoid), natural berry color, chromium (chromium picolinate), and combinations thereof were optionally added to the mixture. After mixing was complete and mixture was homogeneous, an individual serving dose of about 6,000-7,000 mg (e.g., about 6,175 mg) was prepared for administration to the human subject in need thereof. The mixture was further formulated with about 25 to 250 mL of water to give a test formulation. Alternatively, the mixture may be formulated with about 1 to 24 ounces of water to give a test formulation, for example 1 to 2 ounces of water, 2 to 4 ounces of water, 4 to 6 ounces of water, 6 to 8 ounces of water, 8 to 10 ounces of water, 10 to 12 ounces of water, 12 to 18 ounces of water, or 18 to 24 ounces of water.

In another embodiment, about 0.01 g to about 8.99 g of glucomannan (derived from konjac root), about 0.01 to about 8.99 g lemon bioflavonoids, about 0.01 to about 8.99 g resistant potato starch were combined to form a mixture. About 0.1 to about 4.99 g of a polyphenol blend was further added to the mixture. After mixing was complete and mixture was homogeneous, an individual dose of about 9.00 g was prepared for administration to the human subject in need thereof. The mixture was further formulated with about 25 to 250 ml of water to give a test formulation. Alternatively, the mixture may be formulated with about 1 to 24 ounces of water to give a test formulation, for example 1 to 2 ounces of water, 2 to 4 ounces of water, 4 to 6 ounces of water, 6 to 8 ounces of water, 8 to 10 ounces of water, 10 to 12 ounces of water, 12 to 18 ounces of water, or 18 to 24 ounces of water.

### EXAMPLES

### Example 1

25 g amount of glucomannan (Konjac root), 25 g lemon bioflavonoids and 25 g resistant potato starch were combined to form a mixture. 5 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 1.

25 g amount of glucomannan (Konjac root), 10 g lemon bioflavonoids and 10 g resistant potato starch were combined to form a mixture. 5 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 2.

20 g amount of glucomannan (Konjac root), 5 g lemon bioflavonoids and 5 g resistant potato starch were combined to form a mixture. 1 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 3.

15 g amount of glucomannan (Konjac root), 15 g lemon bioflavonoids and 15 g resistant potato starch were combined to form a mixture. 5 g of a polyphenol blend was further added to the mixture. After mixing was complete and mixture was homogeneous, an individual dose of 9 g was prepared for administration to the human subject in need thereof. The mixture was further formulated with 25 to 250 mL of water to give a test formulation 4.

5 g amount of glucomannan (Konjac root), 10 g lemon bioflavonoids and 10 g resistant potato starch were combined to form a mixture. 1 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 5.

15 g amount of glucomannan (Konjac root), 5 g lemon bioflavonoids and 10 g resistant potato starch were combined to form a mixture. 5 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 6.

In formulation 7, glucomannan (Konjac root), lemon bioflavonoids and resistant potato starch were combined in a 1:1:1 ratio by w/w % to form a mixture. 1 to 5 g of a polyphenol blend was further added to the mixture. After mixing was complete and mixture was homogenous, an individual dose of 9 g was prepared for administration to the human subject in need thereof. The mixture was further formulated with 25 to 250 ml of water to give a test formulation 7.

In formulation 8, glucomannan (Konjac root), lemon bioflavonoids and resistant potato starch were combined in a 2:1:1 ratio by w/w % to form a mixture. 1 to 5 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 8.

In formulation 9, glucomannan (Konjac root), lemon bioflavonoids and resistant potato starch were combined in a 2:2:1 ratio by w/w % to form a mixture. 1 to 5 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 9.

In formulation 10, glucomannan (Konjac root), lemon bioflavonoids and resistant potato starch were combined in a 2:2:2 ratio by w/w % to form a mixture. 1 to 5 g of a polyphenol blend was further added to the mixture. After mixing was complete and mixture was homogenous, an individual dose of 9 g was prepared for administration to the human subject in need thereof. The mixture was further formulated with 25 to 250 ml of water to give a test formulation 1.

In formulation 10, glucomannan (Konjac root), lemon bioflavonoids and resistant potato starch were combined in a 4:2:1 ratio by w/w % to form a mixture. 1 to 5 g of a polyphenol blend was further added to the mixture. After mixing was complete and mixture was homogeneous, an individual dose of 9 g was prepared for administration to the human subject in need thereof. The mixture was further formulated with 25 to 250 ml of water to give a test formulation 10.

### Example 2

A test formulation X was prepared according to Table 1 below:

TABLE 1

| Ingredient | mg per serving |
|---|---|
| Solnul (digestive resistant starch from potato) | 3500 |
| Mango Flavor | 325 |
| Peach Flavor | 325 |
| Debitter | 1 |
| Stevia | 90 |
| Citric Acid | 375 |
| Xanthan Gum | 95 |
| Silicon Dioxide | 128.8 |
| Oxxynea (polyphenol blend) | 36 |
| Eriomin (lemon bioflavonoid) | 250 |
| Natural berry color | 50 |
| Glucomannan | 1000 |
| Chromium (from Chromium Picolinate) | 0.023 |

### Example 3

Test formulations 1 through 10 and, X were tested in human subjects for a predetermined period of 7 days.

Data: ≥65% of the time the human subjects experienced a decrease in snacking within seven days after administration. ≥55% of the time the human subjects experienced a decrease in hunger within seven days after administration. ≥50% of the time the human subjects experienced a reduction in cravings for sugar within seven days after administration. ≥40% of the time the human subjects experienced an increase in bowel regularity within seven days after administration.

### Example 4

Test formulation 1 through 10 and, X were tested in humans for a predetermined period of 30 days.

Data: ≥65% of the time the human subjects experienced a decrease in snacking within seven days after administration. ≥55% of the time the human subject experienced a decrease in hunger within seven days after administration. ≥50% of the time the human subjects experienced a reduction in cravings for sugar within seven days after administration. ≥40% of the time the human subjects experienced an increase in bowel regularity within seven days after administration.

≥50% of the time the human subject experiences a reduction in cravings for sugar within thirty days after administration.

≥55% of the time the human subject experiences a decrease in hunger within thirty days after administration.

≥75% of the time the human subject experiences a decrease in snacking within thirty days after administration.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A composition comprising: at least two of the following: a bulking agent; a satiety modifying agent; a prebiotic agent; wherein the composition is formulated in a powder.

Example 2. The composition of any example herein, particularly example 1, wherein the bulking agent comprises glucomannan, cellulose, modified cellulose, hydrogel, psyllium, flax seed, pectin, soluble fiber, or a combination thereof.

Example 3. The composition of any example herein, particularly example 1, wherein the satiety modifying agent comprises lemon bioflavonoids, potato protein, mulberry extract, flavanones, chromium, yeast hydrolysate, lemon verbena, hibiscus, or a combination thereof.

Example 4. The composition of any example herein, particularly example 1, wherein the prebiotic agent comprises resistant potato starch, Akkermansia Muciniphila, pomegranate extract, or a combination thereof.

Example 5. The composition of any example herein, particularly example 1, further comprising one or more optional agents comprising polyphenol blend, amino acids, amino acid metabolites, co-enzyme complex, elemental complex, chelating agents, flavoring agents or a combination thereof.

Example 6. The composition of any example herein, particularly example 5, wherein the coenzyme complex comprises one or more cofactors selected from coenzyme Q10, NADH, NADPH, coenzyme R or B-complex vitamins.

Example 7. The composition of any example herein, particularly example 1, wherein the prebiotic agent comprises one or more additional microbial strains.

Example 8. The composition of any example herein, particularly example 2, wherein the glucomannan is obtained from konjac root.

Example 9. The composition of any example herein, particularly example 2, wherein the soluble fiber is tapioca.

Example 10. The composition of any example herein, particularly example 3, wherein the lemon bioflavonoids comprise eriocitrin.

Example 11. The composition of any example herein, particularly example 1, wherein the bulking agent is present in an amount of about 0.01 g to about 25 g.

Example 12. The composition of any example herein, particularly example 1, wherein the satiety modifying agent is present in an amount of about 0.01 g to about 25 g.

Example 13. The composition of any example herein, particularly example 1, wherein the prebiotic agent is present in an amount of about 0.01 g to about 25 g.

Example 14. The composition of any example herein, particularly example 1, wherein the composition is diary free.

Example 15. The composition of any example herein, particularly example 1, wherein the composition is gluten free.

Example 16. The composition of any example herein, particularly example 1, wherein the composition is sugar free.

Example 17. The composition of any example herein, particularly example 1, further comprising water to form an aqueous solution or suspension.

Example 18. The composition of any example herein, particularly example 17, wherein the water is present is an amount of about 1 to about 24 ounces.

Example 19. A method of inducing satiety in a mammal, comprising administering a composition comprising at least two of the following to the mammal in need thereof: a bulking agent comprising glucomannan optionally derived from konjac root; a satiety modifying agent optionally comprising lemon bioflavonoids; a prebiotic agent comprising potato starch; wherein the individual agents are present in an amount of about 0.01 g to about 25.0 g; and wherein the individual agents are formulated in a powder and mixed with water to form a solution or suspension prior to administration.

Example 20. The method of any example herein, particularly example 19, wherein the composition regulates insulin sensitivity in the mammal.

Example 21. The method of any example herein, particularly example 19, wherein the composition suppresses appetite in the mammal.

Example 22. The method of any example herein, particularly example 19, wherein the composition promotes microbiome in the mammal.

Example 23. The method of any example herein, particularly example 19, wherein the composition induces weight loss in the mammal.

Example 24. The method of any example herein, particularly example 19, wherein the composition promotes GLP1 production in vivo in the mammal.

Example 25. The method of any example herein, particularly example 19, wherein the composition promotes collagen integrity in the mammal.

Example 26. The method of any example herein, particularly example 19, wherein the composition comprising further comprising one or more optional agents comprising polyphenol blend, amino acids, amino acid metabolites, co-enzyme complex, elemental complex, chelating agents, flavoring agents, or a combination thereof.

Example 27. The method of any example herein, particularly example 19, further comprising a polyphenol or polyphenol blend.

Example 28. The method of any example herein, particularly example 19, wherein the mammal is human.

Example 29. The method according to any example herein, particularly any one of examples 19-28, wherein administration is daily for at least 7 days, at least 10 days, or at least 14 days.

Example 30. The method according to any example herein, particularly any one of examples 19-28, wherein administration is twice daily for at least 7 days, at least 10 days, or at least 14 days.

Example 31. The method according to any example herein, particularly any one of examples 19-28, wherein administration is three or more times daily for at least 7 days, at least 10 days, or at least 14 days.

Example 32. The method according to any example herein, particularly any one of examples 19-28, wherein administration is daily for at least 30 days, at least 45 days, at least 60 days, or at least 90 days.

Example 33. The method according to any example herein, particularly any one of examples 19-28, wherein administration is twice daily for at least 30 days, at least 45 days, at least 60 days, or at least 90 days.

Example 34. The method according to any example herein, particularly any one of examples 19-28, wherein administration is three or more times daily for at least 30 days, at least 45 days, at least 60 days, or at least 90 days.

Example 35. A composition comprising: a bulking agent; a satiety modifying agent; and a prebiotic agent; wherein the composition is formulated in a powder.

Example 36. The composition according to any example herein, particularly example 35, wherein the composition comprises a complete absence of organic acids.

Example 37. The composition according to any example herein, particularly any one of examples 35-36, wherein the composition comprises a complete absence of polypeptides.

Example 38. The composition any example herein, particularly any one of examples 35-37, wherein the composition further comprises a pharmaceutically acceptable carrier.

Example 39. The composition according to any example herein, particularly any one of examples 35-38, wherein the composition is suitable for administration to a human subject in need thereof.

Example 40. The composition according to any example herein, particularly any one of examples 35-39, wherein the composition comprises a 0.3 ounce single-use dose.

Example 41. The composition according to any example herein, particularly any one of examples 35-40, wherein the composition comprises a 9 g single-use dose.

Example 42. The composition according to any example herein, particularly any one of examples 35-41, wherein the single-use dose is packaged in a sachet or stick-pack.

Example 43. The composition according to any example herein, particularly any one of examples 35-42, wherein 30 single-use sachets or stick packs are packaged in a box.

Example 44. The composition according to any example herein, particularly any one of examples 35-43, wherein the composition comprises a peach flavoring, mango flavoring, or a combination thereof.

Example 45. The composition according to any example herein, particularly any one of examples 35-44, wherein the satiety modifying agent increases ghrelin, leptin, GLP-1, CCK, PYY, or a combination thereof in the human subject following administration to the human subject.

Example 46. The composition according to any example herein, particularly any one of examples 35-45, wherein the prebiotic promotes the growth of Akkermansia muciniphila in vivo in the human subject following administration to the human subject.

Example 47. The composition according to any example herein, particularly any one of examples 35-46, wherein the prebiotic promotes the growth of Bifidobacterium in vivo in the human subject following administration to the human subject.

Example 48. The composition of any example herein, particularly example 47, wherein the Bifidobacterium comprises Bifidobacterium bifidum, Bifidobacterium infantis, Bifidobacterium longum, or a combination thereof.

Example 49. The composition according to any example herein, particularly any one of examples 35-48, wherein the composition further comprises a polyphenol or polyphenol blend.

Example 50. The composition according to any example herein, particularly any one of examples 35-49, wherein the composition is low FODMOP or suitable for a human subject on a low FODMOP diet.

Example 51. The composition according to any example herein, particularly any one of examples 35-50, wherein the composition is administered to the human subject as a powder, solution, suspension, capsule, drink mix, or drink.

Example 52. The composition according to any example herein, particularly any one of examples 35-51, wherein ≥65% of the time the human subject experiences a decrease in snacking within seven days after administration.

Example 53. The composition according to any example herein, particularly any one of examples 35-52, wherein ≥55% of the time the human subject experiences a decrease in hunger within seven days after administration.

Example 54. The composition according to any example herein, particularly any one of examples 35-51, wherein ≥50% of the time the human subject experiences a reduction in cravings for sugar within seven days after administration.

Example 55. The composition according to any example herein, particularly any one of examples 35-52, wherein ≥40% of the time the human subject experiences an increase in bowel regularity within seven days after administration.

Example 56. The composition according to any example herein, particularly any one of examples 35-55, wherein ≥75% of the time the human subject experiences a decrease in snacking within thirty days after administration.

Example 57. The composition according to any example herein, particularly any one of examples 35-56, wherein ≥55% of the time the human subject experiences a decrease in hunger within thirty days after administration.

Example 58. The composition according to any example herein, particularly any one of examples 35-57, wherein ≥50% of the time the human subject experiences a reduction in cravings for sugar within thirty days after administration.

Example 59. The composition according to any example herein, particularly any one of examples 35-58, wherein the human subject experiences a weight loss of at least 4.1 pounds, at least 4.3 pounds, 4.5 pounds, or 4.7 pounds within thirty days after administration.

Example 60. The composition according to any example herein, particularly any one of examples 35-59, wherein the human subject experiences a weight loss of at least 2.1%, at least 2.2%, at least 2.3%, at least 2.4%, or at least 2.5% of the subject's total body weight within thirty days after administration.

Example 61. A composition suitable for oral administration to a human subject in need thereof comprising: potato starch, glucomannan optionally derived from konjac root, chromium, green olive leaf, grape see extract, green tea extract, grapefruit extract, bilberry extract, white grape extract, red grape extract, and citrus hesperidin.

Example 62. A composition suitable for oral administration to a human subject in need thereof comprising: digestive resistant starch, lemon bioflavonoid, glucomannan, citric acid, chromium, and polyphenol blend.

Example 63. The composition of any example herein, particularly example 62, further comprising silicon dioxide, xanthan gum, or a combination thereof.

Example 64. The composition of any example herein, particularly example 63, further comprising mango flavor, peach flavor, debitter, Stevia, or a combination thereof.

Example 65. A composition suitable for oral administration to a human subject in need thereof comprising: digestive resistant starch from potato, mango flavor, peach flavor, debitter, Stevia, citric acid, xanthan gum, silicon dioxide, Oxxynea (polyphenol blend), Eriomin (lemon bioflavonoid), natural berry color, glucomannan, and chromium (chromium picolinate).

Example 66. A composition suitable for oral administration to a human subject in need thereof comprising a satiety modifying agent, prebiotic agent, and bulking agent, wherein the ratio of satiety modifying agent to prebiotic agent is about 1:5 to about 1:30 by weight, and wherein the ratio of satiety modifying agent to bulking agent is about 1:1 to about 1:10 by weight.

Example 67. A composition described herein are suitable for oral administration to a human subject in need thereof comprising a satiety modifying agent, prebiotic agent, and bulking agent, wherein the ratio of satiety modifying agent to prebiotic agent is about 1:10 to about 1:20 by weight, and wherein the ratio of satiety modifying agent to bulking agent is about 1:2 to about 1:8 by weight.

Example 68. A composition suitable for oral administration to a human subject in need thereof comprising a satiety modifying agent, prebiotic agent, and bulking agent, wherein the ratio of satiety modifying agent to prebiotic agent is about 1:13 to about 1:15 by weight, and wherein the ratio of satiety modifying agent to bulking agent is about 1:3 to about 1:6 by weight.

Example 69. A composition suitable for oral administration to a human subject in need thereof comprising a satiety modifying agent, prebiotic agent, and bulking agent, wherein the ratio of satiety modifying agent to prebiotic agent is about 1:14 to about 1:15 by weight, and wherein the ratio of satiety modifying agent to bulking agent is about 1:4 to about 1:5 by weight.

Example 70. The composition according to any of the previous claims, further comprising mango flavor, peach flavor, debitter, Stevia, citric acid, xanthan gum, silicon dioxide, Oxxynea (polyphenol blend), Eriomin (lemon bioflavonoid), natural berry color, chromium (chromium picolinate), or combinations thereof.

Example 71. The composition according to any example herein, particularly example 70, wherein the mixture is administered with about 25 to 250 ml of water.

Example 72. A method of inducing satiety in a mammal, comprising orally administering at least one serving of composition to the mammal in need thereof, the composition comprising: 3000-4000 mg per serving of prebiotic agent; 750-1250 mg per serving of a bulking agent; and, 100-400 mg per serving of a satiety modifying agent.

Example 73. The method of any example herein, particularly example 72, wherein the bulking agent comprises glucomannan; wherein the satiety modifying agent comprises lemon bioflavonoid; and, the prebiotic agent comprises digestive resistant potato starch.

Example 74. The method of any example herein, particularly example 72, wherein the composition further comprises citric acid, chromium, polyphenol blend, or a combination thereof.

Example 75. The method of any example herein, particularly example 72, wherein the composition further comprises silicon dioxide, xanthan gum, or a combination thereof.

Example 76. The method of any example herein, particularly example 72, wherein the composition further comprises mango flavor, peach flavor, debitter, Stevia, or a combination thereof.

Example 77. The method of any example herein, particularly example 72, wherein the composition regulates insulin sensitivity in the mammal.

Example 78. The method of any example herein, particularly example 72, wherein the composition suppresses appetite in the mammal.

Example 79. The method of any example herein, particularly example 72, wherein the composition promotes microbiome in the mammal.

Example 80. The method of any example herein, particularly example 72, wherein the composition induces weight loss in the mammal.

Example 81. The method of any example herein, particularly example 72, wherein the composition promotes GLP1 production in vivo in the mammal.

Although the invention has been described with reference to the disclosed embodiments, those skilled in the art will readily appreciate that the specific examples and studies detailed above are only illustrative of the invention. It should be understood that various modifications may be made without departing from the spirit of the invention.

The features described herein with regard to any example can be combined with other features described in any one or more of the other examples, unless otherwise stated.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A composition comprising:
at least two of the following:
a bulking agent;
a satiety modifying agent;
a prebiotic agent; wherein the composition is formulated in a powder.

2. The composition of claim 1, wherein the bulking agent comprises glucomannan, cellulose, modified cellulose, hydrogel, psyllium, flax seed, pectin, soluble fiber, or a combination thereof.

3. The composition of claim 2, wherein the glucomannan is obtained from konjac root, wherein the soluble fiber is tapioca, and wherein the composition further comprises lemon bioflavonoids comprising eriocitrin.

4. The composition of claim 1, wherein the satiety modifying agent comprises lemon bioflavonoids, potato protein, mulberry extract, flavanones, chromium, yeast hydrolysate, lemon verbena, hibiscus, or a combination thereof.

5. The composition of claim 1, wherein the prebiotic agent comprises resistant potato starch, Akkermansia Muciniphila, pomegranate extract, or a combination thereof.

6. The composition of claim 1, further comprising one or more optional agents comprising polyphenol blend, amino acids, amino acid metabolites, co-enzyme complex, elemental complex, chelating agents, flavoring agents or a combination thereof.

7. The composition of claim 6, wherein the co-enzyme complex comprises one or more cofactors selected from coenzyme Q10, NADH, NADPH, coenzyme R or B-complex vitamins.

8. The composition of claim 1, wherein the bulking agent is present in an amount of about 0.01 g to about 25 g, the satiety modifying agent is present in an amount of about 0.01 g to about 25 g, and/or the prebiotic agent is present in an amount of about 0.01 g to about 25 g.

9. The composition of claim 1, further comprising water to form an aqueous solution or suspension, wherein the water is present is an amount of about 1 to about 24 ounces.

10. A method of inducing satiety in a mammal, comprising administering a composition comprising at least two of the following individual agents to the mammal in need thereof:
a bulking agent comprising glucomannan optionally derived from konjac root;
a satiety modifying agent optionally comprising lemon bioflavonoids;
a prebiotic agent comprising potato starch;
wherein the individual agents are present in an amount of about 0.01 g to about 25.0 g; and
wherein the individual agents are formulated in a powder and mixed with water to form a solution or suspension prior to administration.

11. The method of claim 10, wherein the composition regulates insulin sensitivity in the mammal, suppresses appetite in the mammal, promotes microbiome in the mammal, induces weight loss in the mammal, promotes GLP1 production in vivo in the mammal, and/or promotes collagen integrity in the mammal.

12. The method of claim 10, wherein administration is 1, 2, 3, or more times daily for at least 30 days, at least 45 days, at least 60 days, or at least 90 days.

13. A composition for administration to a human subject in need thereof, the composition comprising:
a bulking agent;
a satiety modifying agent; and
a prebiotic agent; wherein the composition is formulated in a powder.

14. The composition according to claim 13, wherein the composition comprises a complete absence of organic acids and/or polypeptides.

15. The composition according to claim 13, wherein the composition further comprises a pharmaceutically acceptable carrier, a peach flavoring, a mango flavoring, a polyphenol, a polyphenol blend, or a combination thereof.

16. The composition according to claim 13, wherein the composition comprises a 0.3 ounce single-use dose or a 9 g single-use dose.

17. The composition according to claim 13, wherein the prebiotic promotes growth of Akkermansia muciniphila in vivo in the human subject following administration to the human subject, and/or promotes growth of Bifidobacterium in vivo in the human subject following administration to the human subject.

18. The composition of claim 13, wherein the composition comprises:
potato starch, glucomannan optionally derived from konjac root, chromium, green olive leaf, grape see extract, green tea extract, grapefruit extract, bilberry extract, white grape extract, red grape extract, and citrus hesperidin.

19. The composition of claim 13, wherein the composition comprises:
digestive resistant starch, lemon bioflavonoid, glucomannan, citric acid, chromium, polyphenol blend; and
one or more of silicon dioxide, xanthan gum, mango flavor, peach flavor, debitter, and Stevia.

20. The composition of claim 13, the composition comprising:
3000-4000 mg per serving of the prebiotic agent;
750-1250 mg per serving of the bulking agent; and,
100-400 mg per serving of the satiety modifying agent.
